(19) United States
(12) Patent Application Publication
(10) Pub. No.: US 2026/0186120 A1
(43) Pub. Date: Jul. 2, 2026

(54) mmWave Radar System using Unified Artificial Intelligence Model

(71) Applicant: KaiKuTek INC., Taipei (TW)

(72) Inventors: Tsung-Ming Tai, Taipei (TW); Mike Chun-Hung Wang, Taipei (TW); Wen-Jyi Hwang, Taipei (TW); Wei-Chi Li, Taipei (TW); Kuan-Yu Liu, Taipei (TW)

(73) Assignee: KaiKuTek INC., Taipei (TW)

(21) Appl. No.: 19/006,254

(22) Filed: Dec. 31, 2024

(51) Int. Cl. G01S 13/04; A61B 5/0507; G01S 13/89

(52) U.S. Cl. CPC G01S 13/04; A61B 5/0507; G01S 13/89

(57) ABSTRACT

An mmWave radar system includes an mmWave radar sensor and a unified artificial intelligence (AI) model. The unified AI model includes an AI encoder, and an AI decoder. The mmWave radar sensor is used to detect environmental data. The AI encoder is coupled to the mmWave radar sensor for compressing the environmental data to generate a unified representation. The AI decoder is coupled to the AI encoder for analyzing the unified representation for generating a plurality of task results.

### BACKGROUND OF THE INVENTION

### 1. Field Of The Invention

The present invention is related to an mmWave radar system, in particularly related to an mmWave radar system using a unified artificial intelligence model.

### 2. Description Of The Prior Art

Ambient sensing refers to technology that collects data from the surrounding environment. This information can be utilized for various purposes, including understanding environmental conditions, providing users with relevant information, and controlling devices.

In ambient sensing, a network of sensors collaborates to gather and provide information. Although almost any sensor can theoretically be used as an ambient sensor, the most common types include temperature sensors, pressure sensors, water sensors, and object sensors.

Temperature sensors can continuously monitor the temperatures within a home, providing average readings that indicate whether the home maintains a healthy temperature. Pressure sensors, in conjunction with temperature sensors, can help form a comprehensive picture of the weather conditions around the home. Water sensors can detect increased humidity levels indoors. Object sensors, equipped with radio frequency identification (RFID) tags or global positioning system (GPS) trackers, can be attached to key items or individuals to gather insights on usage and movement.

However, traditional ambient or environmental sensors have limited utility due to their simple outputs. In contrast, an mmWave radar sensor provides comprehensive radar signals for ambient sensing. These complete radar signals can be analyzed using a unified artificial intelligence model to perform a variety of tasks.

### SUMMARY OF THE INVENTION

An embodiment provides an mmWave radar system. The mmWave radar system includes an mmWave radar sensor and a unified artificial intelligence (AI) model. The unified AI model includes an AI encoder, and an AI decoder. The mmWave radar sensor is used to detect environmental data. The AI encoder is coupled to the mmWave radar sensor for compressing the environmental data to generate a unified representation. The AI decoder is coupled to the AI encoder for analyzing the unified representation for generating a plurality of task results.

An embodiment provides an mmWave radar system. The mmWave radar system includes an mmWave radar sensor, a digital signal processor, and a unified artificial intelligence (AI) model. The unified AI model includes an AI encoder, and an AI decoder. The mmWave radar sensor is used to detect environmental data. The digital signal processor is coupled to the mmWave radar sensor for performing digital signal preprocessing on the environmental data to generate preprocessed data. The AI encoder is coupled to the digital signal processor for compressing the preprocessed data to generate a unified representation. The AI decoder is coupled to the AI encoder for analyzing the unified representation to generate a plurality of task results.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a block diagram of an mmWave radar system according to an embodiment of the present invention.

FIGS. 2A to 2D are schematic diagrams of task results performed by an artificial intelligence (AI) model of the mmWave radar system in FIG. 1.

FIG. 3 is a block diagram of an mmWave radar system using a unified AI model according to an embodiment of the present invention.

FIG. 4 is a block diagram of an mmWave radar system using a unified AI model according to an embodiment of the present invention.

### DETAILED DESCRIPTION

Millimeter wave (mmWave) sensing is a non-contact technology that uses mmWave radar sensors to measure movement, acceleration, and angles with precision down to a fraction of a millimeter. This system operates by transmitting and receiving pulses of millimeter electromagnetic wave energy, detecting targets and motion from the reflected signals. Additional components such as converters, signal processors, and other embedded technologies enhance the system's performance and enable new applications. Current uses of this technology include tracking human and animal movement, detecting human presence, and monitoring vital signs. These applications span various industries, including automotive, meteorological, medical, and pet health, often serving as an alternative to wearable-based technologies.

Compared to other radio frequency sensing technologies in the electromagnetic spectrum, such as infrared or ultra-wideband, mmWave operates in the 10 to 100 gigahertz (GHz) range. Typical mmWave sensors utilize the 24 GHz, 60 GHz, and 77 GHz bands, each offering unique advantages for specific applications.

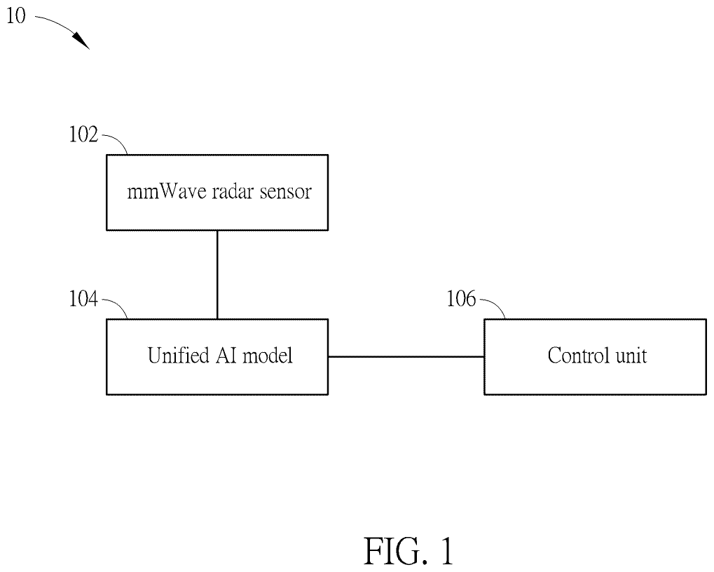

FIG. 1 is a block diagram of an mmWave radar system 10 according to an embodiment of the present invention. The mmWave radar system 10 may comprise an mmWave radar sensor 102, a unified artificial intelligence (AI) model 104, and a control unit 106. The mmWave radar sensor 102, the unified AI model 104, and the control unit 106 may be disposed inside an interactive device. The unified AI model 104 may be coupled to the mmWave radar sensor 102 and the control unit 106. The mmWave radar sensor 102 is used to detect environmental data, and the unified AI model 104 is used to process the environmental data and generate a plurality of task results. The unified AI model 104 may comprise an AI encoder and an AI decoder. The AI encoder is trained to adapt the radar input data into a unified representation for multi-tasking. The AI decoder learns to utilize the unified representation and decode it into the multi-task results. The control unit 106 operates the interactive device according to the plurality of task results. In an embodiment, the environmental data includes heart rate, respiratory rate, gesture, posture, position and/or velocity of a living being. In another embodiment, the environmental data includes position and/or velocity of a non-living object or a plant.

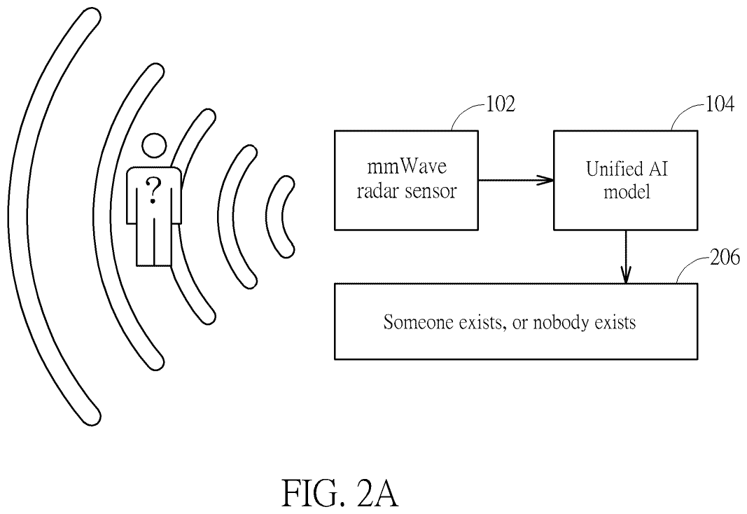

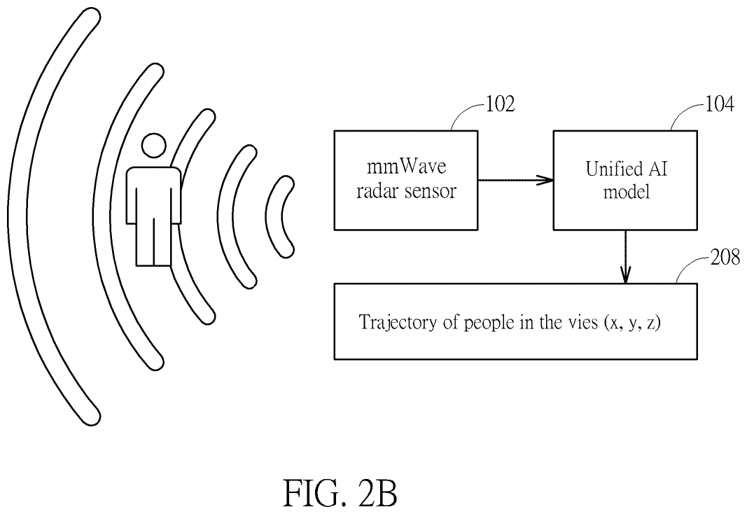

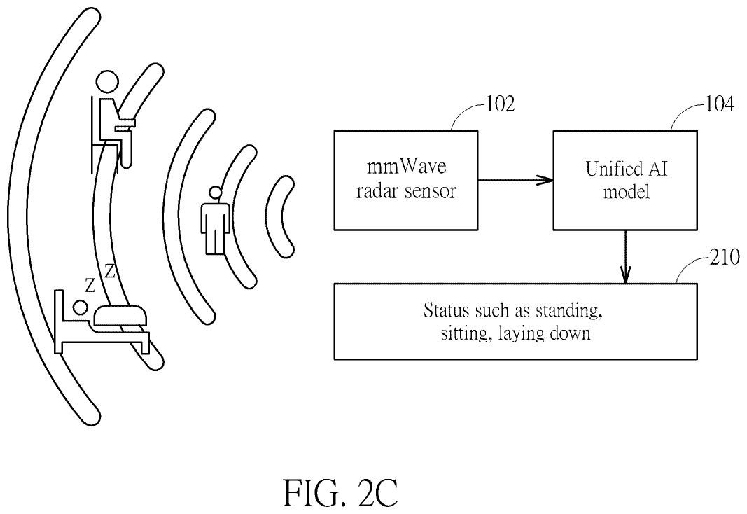

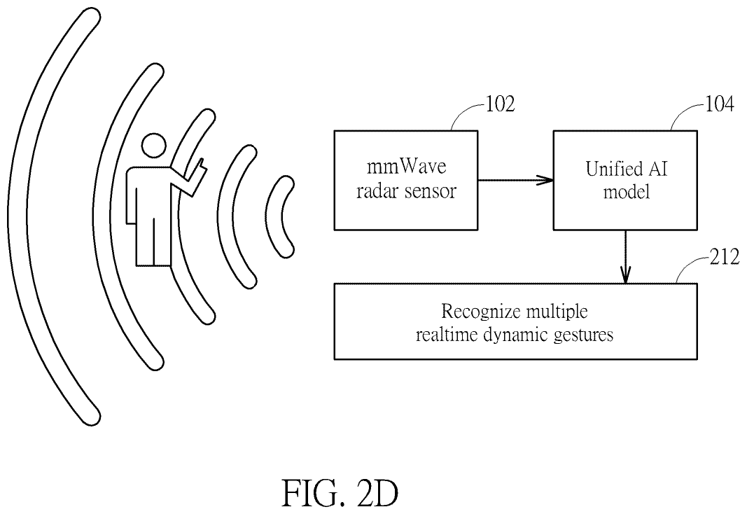

FIGS. 2A to 2D are schematic diagrams of task results performed by the unified artificial intelligence (AI) model 104. In one embodiment, the mmWave radar sensor 102 detects environmental data to generate an mmWave radar signal. This environmental data can include heart rate, respiratory rate, gestures, posture, and the position and/or velocity of a living being. In another embodiment, the data includes the position and/or velocity of a non-living object or a plant. An unified AI model 104 analyzes the mmWave radar signal to generate various task results, which can include presence detection 206, people tracking 208, posture detection 210, and gesture recognition 212. For presence detection 206, the unified AI model 104 determines whether someone is present. For people tracking 208, the unified AI model 104 tracks a person's position in three-dimensional space (x, y, z). For posture detection 210, the unified AI model 104 identifies the posture of a person, such as standing, sitting, or sleeping. For gesture recognition 212, the unified AI model 104 recognizes various real-time gestures.

As mentioned above, the unified AI model 104 may generate various task results, such as presence detection 206, people tracking 208, posture detection 210, and gesture recognition 212. The unified AI model 104 can perform a single task or multiple tasks simultaneously. The unified AI model 104 may comprise an AI encoder and an AI decoder. The AI encoder is trained to adapt the radar input data into a unified representation for multi-tasking. The AI decoder learns to utilize the unified representation and decode it into the multi-task results. The control unit 106 may operate the interactive device based on these task results. Examples of interactive devices include smart fans, smart televisions, spatial audio systems, air conditioners, smart lighting, security surveillance system, and electric doors. In an embodiment, the mmWave radar sensor 102 transmits a plurality of frequency modulated continuous wave (FMCW) signals and receives a plurality of reflected FMCW signals. The unified AI model 104 analyzes the plurality of reflected signals to generate the task results.

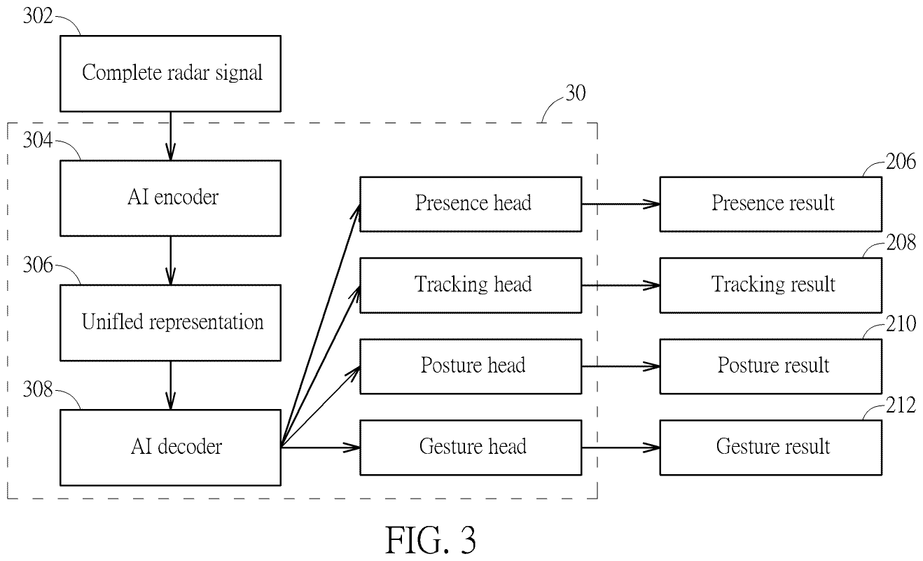

FIG. 3 is a block diagram of a unified AI model 30 according to an embodiment of the present invention. The unified AI model 30 includes an AI encoder 304, and an AI decoder 308. In an embodiment, the mmWave radar sensor 102 may transmit a frequency modulated continuous wave (FMCW) signal and receive a plurality of reflected FMCW signals 302. The AI encoder 304 compresses the plurality of reflected FMCW signals 302 to generate a unified representation 306. The AI decoder 308 analyzes the unified representation 306 to generate a plurality of task results 206, 208, 210, 212. The task results include presence result 206, tracking result 208, posture result 210, and gesture result 212. For presence result 206, the AI decoder 308 analyzes the unified representation 306 to determine whether someone exists or not. For tracking result 208, the AI decoder 308 analyzes the unified representation 306 to track a person with a position of (x, y, z). For posture result 210, the AI decoder 308 analyzes the unified representation 306 to determine a posture of a person such as sitting, standing, or sleeping. For gesture result 212, the AI decoder 308 analyzes the unified representation 306 to recognize a real-time dynamic gesture and respond to the gesture.

In an embodiment, the AI encoder 304 and the AI decoder 308 comprise a plurality of learnable weights (parameters) that can be adjustable by a data-driven training process that is directly optimized based on the task results. The AI encoder 304 and the AI decoder 308 are constructed using the multi-layers of learnable parameters which comprises (including but not limited to) 1-D or 2-D convolutions, matrix multiplication, and non-parameterized operators comprising shape manipulate operators (transpose, reshape, etc.,) and element-wise activation functions comprising ReLU, Sigmoid, hyperbolic tangent, etc.

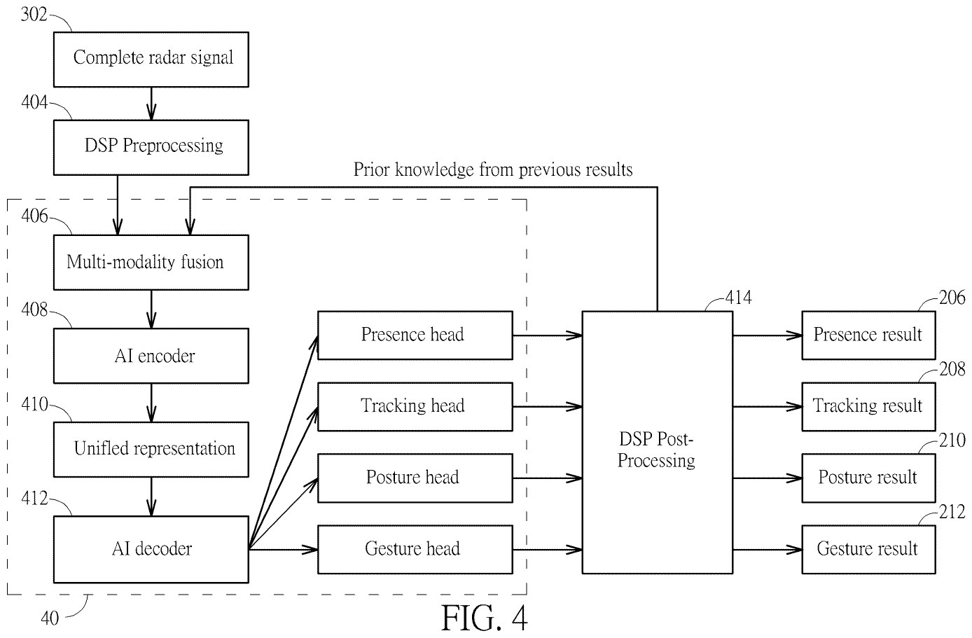

FIG. 4 is a block diagram of a unified AI model 40 according to an embodiment of the present invention. The unified AI model 40 includes an AI encoder 408, and an AI decoder 412. In one embodiment, the mmWave radar sensor 102 may transmit a frequency modulated continuous wave (FMCW) signal and receive a plurality of reflected FMCW signals 302. A digital signal processor performs digital signal preprocessing 404 on the plurality of reflected FMCW signals 302 to generate preprocessed data. This digital signal preprocessing 404 may include fast Fourier transform (FFT), digital Fourier transform (DFT) decimation, wavelet transform, and/or point-cloud analysis. The AI encoder 408 compresses the preprocessed data to create a unified representation 410. The AI decoder 412 then analyzes this unified representation 410 to generate various task results 206, 208, 210, 212, including presence detection 206, people tracking 208, posture detection 210, and gesture recognition 212.

In an embodiment, the AI encoder 408 and the AI decoder 412 comprise a plurality of learnable weights (parameters) that can be adjustable by a data-driven training process that is directly optimized based on the task results. The AI encoder 408 and the AI decoder 412 are constructed using the multi-layers of learnable parameters which comprises (including but not limited to) 1-D or 2-D convolutions, matrix multiplication, and non-parameterized operators comprising shape manipulate operators (transpose, reshape, etc.,) and element-wise activation functions comprising ReLU, Sigmoid, hyperbolic tangent, etc.

In an embodiment, the task results 206, 208, 210, 212 generated by the AI decoder 412 are digital signal post-processed by the digital signal processor to generate post-processed results. The digital signal post-processing 414 may include Kalman filtering, particle filtering, and/or clustering. The post-processed results may include presence result 206, tracking result 208, posture result 210, and gesture result 212. In an embodiment, the post-processed results can be used to combine with the preprocessed data in next turn and the fusion is compressed by the AI encoder 408 to generate a unified representation 410. In other words, the prior post-processed results can be used with the preprocessed data to enhance the analysis of the unified representation 410.

In one embodiment, the orientation of the interactive device is adjusted based on the task results 206, 208, 210, 212 generated by the AI decoder 412. For example, if the interactive device is a smart fan, the mmWave radar system 10 detects a person's position as a tracking result 208 and adjusts the fan to face the person. Similarly, if the interactive device is a spatial audio system, the mmWave radar system 10 detects a person's position as a tracking result 208 and adjusts the audio to ensure it surrounds the person. Additionally, the mmWave radar system 10 can detect the position of non-living objects or plants and adjust the interactive device accordingly.

In an embodiment, the control unit 106 turns on or off the interactive device according to the task results 206, 208, 210, 212 generated by the AI decoder 412. For example, the interactive device is a smart television, and the unified AI model 40 detects the posture and/or gesture of a person as a posture result 210 and/or a gesture result 212. The control unit 106 may adjust or turn on/off the smart television according to the posture result 210 and/or the gesture result 212. In another example, the interactive device is an electric door, and the unified AI model 40 generates the posture result 210 and/or a gesture result 212. The control unit 106 may open or close the electric door according to the posture result 210 and/or the gesture result 212. In another example, the interactive device is an air conditioner, and the unified AI model 40 generates the posture result 210 and/or the gesture result 212. The control unit 106 may adjust or turn on/off the air conditioner according to the posture result 210 and/or the gesture result 212. In another embodiment, the unified AI model 40 detects the position and/or movement of a non-living object or a plant as a tracking result 208, and the control unit 106 may turn on/off the interactive device according to the position and/or movement of the non-living object or a plant.

In summary, the present invention provides an mmWave radar system 10 using a unified AI model 30, 40 for presence detection, people tracking, posture detection, and/or gesture recognition. The unified AI model 30, 40 performs better than the prior art using a unified representation 410 and can be applied to perform presence detection, people tracking, posture detection, and/or gesture recognition.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An mmWave radar system, comprising:
an mmWave radar sensor, configured to detect environmental data; and
a unified artificial intelligence (AI) model comprising:
an AI encoder, coupled to the mmWave radar sensor, and configured to compress the environmental data to generate a unified representation; and
an AI decoder, coupled to the AI encoder, and configured to analyze the unified representation for generating a plurality of task results.

2. The mmWave radar system of claim 1, wherein the plurality of task results comprise a presence detection result, an object and user tracking result, a posture analysis result, and/or a gesture recognition result.

3. The mmWave radar system of claim 1, wherein the environmental data comprises heart rate, respiratory rate, gesture, posture, position and/or velocity of a living being.

4. The mmWave radar system of claim 1, wherein the environmental data comprises position and/or velocity of a non-living object.

5. The mmWave radar system of claim 1, further comprising:
an interactive device, coupled to the AI decoder, configured to respond according to the plurality of task results.

6. The mmWave radar system of claim 5, wherein the interactive device is a smart fan, a smart television, a spatial audio, an air conditioner, smart lighting, security surveillance system, or an electric door.

7. The mmWave radar system of claim 1, wherein the mmWave radar sensor transmits a plurality of frequency modulated continuous wave (FMCW) signals and receives a plurality of reflected FMCW signals.

8. The mmWave radar system of claim 7, wherein the AI encoder compresses the plurality of reflected FMCW signals to generate a unified representation.

9. An mmWave radar system, comprising:
an mmWave radar sensor, configured to detect environmental data;
a digital signal processor, coupled to the mmWave radar sensor, and configured to perform digital signal preprocessing on the environmental data to generate preprocessed data; and
a unified artificial intelligence (AI) model comprising:
an AI encoder, coupled to the digital signal processor, configured to compress the preprocessed data to generate a unified representation; and
an AI decoder, coupled to the AI encoder, configured to analyze the unified representation to generate a plurality of task results.

10. The mmWave radar system of claim 9, wherein the plurality of task results comprise a presence detection result, an object and user tracking result, a posture analysis result, and/or a gesture recognition result.

11. The mmWave radar system of claim 9, wherein the environmental data comprises heart rate, respiratory rate, gesture, posture, position and/or velocity of a living being.

12. The mmWave radar system of claim 9, wherein the environmental data comprises position and/or velocity of a non-living object.

13. The mmWave radar system of claim 9, further comprising:
an interactive device, coupled to the AI decoder, configured to respond according to the plurality of task results.

14. The mmWave radar system of claim 13, wherein the interactive device is a smart fan, a smart television, a spatial audio, an air conditioner, smart lighting, security surveillance system, or an electric door.

15. The mmWave radar system of claim 9, wherein the mmWave radar sensor transmits a plurality of frequency modulated continuous wave (FMCW) signals and receives a plurality of reflected FMCW signals.

16. The mmWave radar system of claim 15, wherein the digital signal processor preprocesses the plurality of reflected FMCW signals to generate the preprocessed data.

17. The mmWave radar system of claim 9, wherein the digital signal processor performs digital signal post-processing on the plurality of task results to generate a plurality of post-processed results.

18. The mmWave radar system of claim 17, wherein the digital signal post-processing comprises kalman filtering, particle filtering, and/or clustering.

19. The mmWave radar system of claim 17, wherein the AI encoder compresses fusion of the preprocessed data and a plurality of prior post-processed results to generate the unified representation.

20. The mmWave radar system of claim 9, wherein the digital signal preprocessing comprises fast Fourier transform (FFT), digital Fourier transform (DFT) decimation, wavelet transform, and/or point-cloud analysis.
